(19) United States
(12) Patent Application Publication
(10) Pub. No.: US 2022/0059209 A1
(43) Pub. Date: Feb. 24, 2022

(54) DEVICE, SYSTEM, AND METHOD FOR PROVIDING TREATMENT INFORMATION FOR SKIN BEAUTY TREATMENT

(71) Applicant: HIRONIC CO., LTD., Youngin-si, Gyeonggi-do (KR)

(72) Inventors: Jin Woo WOO, Yongin-si, Gyeonggi-do (KR); Ha Gyeong SEONG, Yongin-si, Gyeonggi-do (KR); In Geom JEON, Wonju-si Gangwon-do (KR)

(73) Assignee: HIRONIC CO., LTD., Youngin-si, Gyeonggi-do (KR)

(21) Appl. No.: 17/299,342

(22) PCT Filed: Dec. 4, 2019
(86) PCT No.: PCT/KR2019/016969

§ 371 Date: Jun. 3, 2021

(30) Foreign Application Priority Data
Dec. 4, 2018 (KR) 10-2018-0154138

(51) Int. Cl. G16H 20/40; A61B 5/00; A61B 5/103; A61N 5/06; G16H 40/67; G16H 10/60; G06T 7/00

(52) U.S. Cl. CPC G16H 20/40; A61B 5/441; A61B 5/1032; A61N 5/0616; G16H 40/67; G16H 10/60; G06T 2207/10081; A61N 2005/0661; A61N 2005/0626; G06T 2207/30088; G06T 2207/10088; G06T 2207/10132; G06T 7/0012

(57) ABSTRACT

Disclosed are a device, a system, and a method of providing treatment information for a skin care treatment. The device includes: a patient information providing unit configured to provide patient information; a treatment information generating unit configured to generate treatment information based on the patient information; a progress information input unit configured to receive progress information for the treatment when the treatment is completed based on the treatment information; and a control unit configured to update the treatment information based on the progress information. Therefore, it is possible to provide the skin care treatment in a more objective state by preventing the treatment from being performed under different conditions for each practitioner or according to a condition of the practitioner, and it is further possible to perform a safe and effective treatment by preventing misuse of medical technology due to the misjudgment of the practitioner.

### TECHNICAL FIELD

The present invention relates to a device, a system, and a method of providing treatment information for a skin care treatment, and more particularly, to a device, a system, and a method of providing treatment information for a skin care treatment, which are capable of presenting an optimized treatment method for each patient, lesion, and part in consideration of a skin condition and treatment progress.

### BACKGROUND ART

Skin care refers to maintaining, protecting, and improving the skin beautifully by supplying nutrition by enhancing a physiological function of the skin and activating the metabolism by treating a face and whole body skin by using physical and chemical methods.

The purpose of the skin care is to restore the physiological function of the skin to maintain the skin to be healthful and elastic, and help find the skin's unique characteristics and properties to improve the skin to normal skin and maintain healthy skin.

A method of the skin care may include a method of protecting, maintaining, and improving the skin by using cosmetics, manual techniques, and the like, and a method of improving lifestyle, eating habits, and skin care habits, and the like, and recently, there are increasing cases where people expect more direct and effective improvement effects by receiving care through a skin care treatment in skin clinics.

In the meantime, in the skin care treatment, it is effective to differentiate the skin condition, the treatment area, and lesion that the patient has, and change setting values of tools and devices used in the skin care treatment. For example, in the case of focused ultrasound lifting treatment, the treatment is performed by targeting the fascia layer, so that the setting value may be changed according to a skin thickness of a patient.

However, the tools and devices used in the skin care treatment are changed or adjusted and used according to the subjective judgement of a practitioner, so that the improvement effect according to the skin care is inevitably influenced by the subjectivity of the practitioner.

Accordingly, it is requested to find a method to ensure a safe and effective procedure by presenting a more objective method for the skin care treatment to prevent misuse of the subjective judgement of the practitioner.

### DISCLOSURE

### Technical Problem

The present invention is conceived to solve the foregoing problems, and the object of the present invention is to provide a device, a system, and a method of providing treatment information for a skin care treatment, which are capable of objectively presenting an optimized treatment method for each patient, lesion, and part in consideration of a skin condition and treatment progress of a patient.

### Technical Solution

In order to achieve the object, a device for providing treatment information according to an exemplary embodiment of the present invention includes: a patient information providing unit configured to provide patient information; a treatment information generating unit configured to generate treatment information based on the patient information; a progress information input unit configured to receive progress information for the treatment when the treatment is completed based on the treatment information; and a control unit configured to update the treatment information based on the progress information.

Herein, the patient information providing unit may include: a biometric information providing unit configured to receive and provide biometric information of a patient as the patient information; and a processed information providing unit configured to generate and provide first skin information that is analyzed based on the biometric information, second skin information obtained by photographing a skin of the patient, and third skin information obtained by radiating light to the skin of the patient as the patient information.

Further, the first skin information may be at least one of skin thickness information classified according to a Body Mass Index (BMI) in the biometric information and skin color information classified according to Fitzpatrick skin type in the biometric information, the second skin information may be skin thickness information measured through at least one device among an ultrasonic photographing device, a Computed Tomography (CT) device, and a Magnetic Resonance Imaging (MRI) device, and the third skin information may be skin lesion information for each part obtained through a skin analysis through ultraviolet light or polarized light radiation.

In addition, the treatment information generating unit may generate the treatment information for treating a patient before a skin treatment based on the skin thickness information, the skin color information, and the skin lesion information, and the treatment information may be generated for each patient, lesion, and part, and is provided to a practitioner, and is at least one of handpiece information, cartridge information, pin type information, treatment intensity information, delay time information, cooling condition information, spot size information, frequency information, and period information which are determined to be most suitable for the selected patient, selected lesion, and selected part and may be provided in a modifiable state.

Further, the device for providing treatment information according to the present exemplary embodiment may further include: an internal access processing unit configured to process log-in of the practitioner; and a remote access processing unit configured to process log-in using the biometric information of the patient through an external terminal.

In addition, the progress information may include primary progress information based on satisfaction input from the practitioner or a patient after the treatment is completed, and secondary progress information based on a skin state change input from the practitioner or the patient before an additional treatment after a predetermined time elapses.

Further, the treatment information may be generated for each patient, lesion, and part and be provided to a practitioner, and be at least one of handpiece information, cartridge information, pin type information, treatment intensity information, delay time information, cooling condition information, spot size information, frequency information, and period information which are determined to be most suitable for the selected patient, selected lesion, and selected part and be provided in a modifiable state, the primary progress information and the secondary progress information may be input for each patient, lesion, and part, and the control unit may update the treatment information for each patient, lesion, and part based on the input first progress information and the input second progress information and provide the updated information in a modifiable state for the additional treatment.

Further, the device for providing treatment information according to the present exemplary embodiment may further include: a storage unit configured to store the primary progress information and at least one secondary progress information continuously generated when the additional treatment is repeated; and a communication unit configured to allow the primary progress information and at least one secondary progress information stored in the storage unit to be shared with an external treatment information providing device used by another practitioner.

In the meantime, in order to achieve the object, a system for providing treatment information according to an exemplary embodiment of the present invention includes: at least one treatment information providing device configured to generate treatment information based on patient information, receive progress information for the treatment, update the treatment information, and provide the updated treatment information; a server configured to store and update the patient information, the treatment information, and the progress information received from at least one treatment information providing device; and a patient terminal provided to generate the progress information by accessing the server by using biometric information in the patient information.

In the meantime, in order to achieve the object, a method of providing treatment information according to an exemplary embodiment of the present invention includes: receiving patient information; generating treatment information based on the patient information; receiving progress information for the treatment when the treatment is completed based on the treatment information; and updating the treatment information based on the progress information and providing the updated treatment information.

### Advantageous Effects

Therefore, it is possible to provide the skin care treatment in a more objective state by preventing the treatment from being performed under different conditions for each practitioner or according to a condition of the practitioner, and it is further possible to perform a safe and effective treatment by preventing misuse of medical technology due to the misjudgment of the practitioner.

### DESCRIPTION OF DRAWINGS

FIG. 1 is a diagram illustrating a system for providing treatment information for a skin care treatment according to an exemplary embodiment of the present invention.

FIG. 2 is a block diagram illustrating a configuration of a device for providing treatment information according to the exemplary embodiment of the present invention.

FIG. 3 is a diagram illustrating a screen in which patient information is provided according to the exemplary embodiment of the present invention.

FIG. 4 is a diagram illustrating a screen in which treatment information is provided according to the exemplary embodiment of the present invention.

FIG. 5 is a diagram illustrating a screen in which progress information is input according to the exemplary embodiment of the present invention.

FIG. 6 is a diagram illustrating a screen in which a treatment history is recorded according to the exemplary embodiment of the present invention.

FIG. 7 is a diagram illustrating a method of providing treatment information for a skin care treatment according to an exemplary embodiment of the present invention.

### BEST MODE

Hereinafter, the present invention will be described in more detail with reference to the drawings. Exemplary embodiments introduced hereinafter are provided as examples for sufficiently transferring the spirit of the present invention to those skilled in the art. The present invention is not limited to the exemplary embodiments described below and may be embodied in other forms.

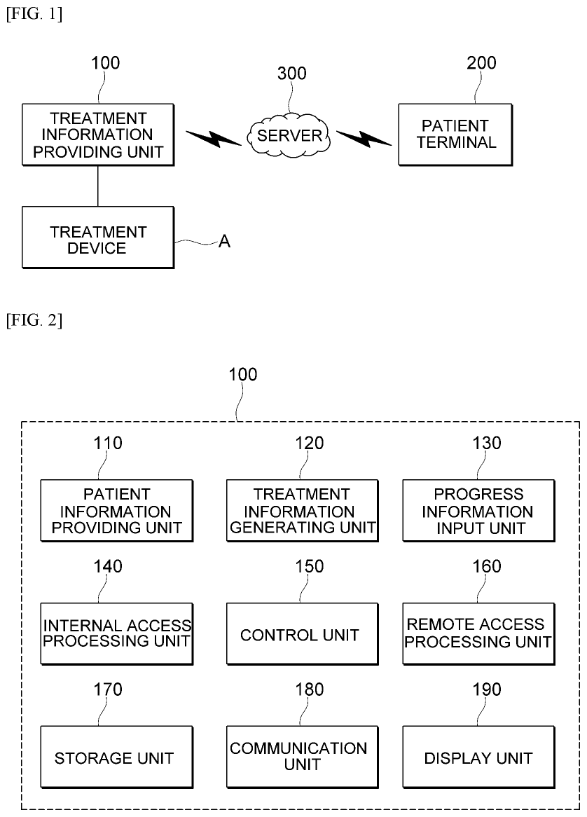

FIG. 1 is a diagram illustrating a system for providing treatment information for a skin care treatment according to an exemplary embodiment of the present invention.

The system for providing treatment information according to the exemplary embodiment provides a treatment method to enable an objective treatment for each patient, lesion, and part according to a condition of a patient so that subjective judgement of a practitioner is minimized during a skin cosmetic treatment.

To this end, the system for providing treatment information according to the exemplary embodiment includes a treatment information providing device 100, a patient's terminal 200, and a server 300.

The treatment information providing device 100 provides treatment information to enable an objective treatment based on basic information of a patient, and updates the treatment information for a later treatment based on progress information after the treatment is completed.

The treatment information providing device 100 serves to drive a treatment device A according to the provided treatment information or treatment information corrected by a practitioner.

The treatment information providing device 100 transmits the input information, the processed information, and the updated information to the server 300 and makes the transmitted information be stored in the server 300.

The patient's terminal 200 serves to read the information input, processed, or updated in the treatment information providing device 100 through the server 300. To this end, the patient's terminal 200 may be configured with a medium, such as a smart phone, capable of performing wireless communication.

Particularly, the patient's terminal 200 may be configured with a medium, in which an application program distributed in a treatment center and the like in which the treatment is performed is installable. That is, in this case, the patient's terminal 200 may read the information input, processed, or updated in the treatment information providing device 100 by accessing the server 300 after installing the application program.

In the meantime, the patient's terminal 200 serves to input progress information for an evaluation of the treatment after the treatment is completed. After the treatment is completed, the progress of the treatment may be input by the practitioner, but may also be input ty the patient. As described above, when the progress information for the treatment is input by the patient, the patient may input the progress information through the treatment information providing device 100 prepared in the treatment center, but may also input the progress information through the personally possessed patient's terminal 200.

As described above, the information input, processed, or updated in the treatment information providing device 100 may be read through the external patient's terminal 200 through the server 300, and separate information may be input through the external patient's terminal 200, so that the patient may have confidence in the treatment of the practitioner and the practitioner may perform the treatment by a transparent treatment method according to disclosure.

Hereinafter, the configurations for providing the objective treatment method by the treatment information providing device 100 will be described.

FIG. 2 is a block diagram illustrating the configuration of the treatment information providing device 100 according to the exemplary embodiment of the present invention.

The treatment information providing device 100 according to the present exemplary embodiment serves to receive patient information required for a treatment, generate treatment information for the treatment based on the received patient information, receive progress information after the treatment is completed, and update the treatment information after a later treatment.

To this end, the treatment information providing device 100 according to the present exemplary embodiment includes a patient information providing unit 110, a treatment information generating unit 120, a progress information input unit 130, an internal access processing unit 140, a control unit 150, a remote access processing unit 150, a storage unit 170, a communication unit 180, and a display unit 190.

The patient information providing unit 110 serves to receive or generate and provide patient information through various means.

Herein, the patient information is the concept including not only biometric information related to the body, such as height, weight, age, gender, voice, iris, fingerprint, and face shape of the patient, but also first skin information analyzed from the biometric information, and second and third skin information obtained through separate devices.

Particularly, the biometric information itself may also be used for identifying a patient, but may be processed to the first skin information required for the skin care treatment through the analysis of the biometric information.

For example, when information on age, weight, and height is input as the biometric information, the patient information providing unit 110 may provide the information on age, weight, and height to the treatment information generating unit 120 which is to be described below, but separately, the patient information providing unit 110 may also generate the first skin information regarding whether the patient's skin is thin or thick by calculating the obesity rate against the appropriate Body Mass Index (BMI) for each age by using the information on age, weight, and height.

When it is determined that the skin condition is 20% thicker than a reference value through the generated first skin information, the setting values for the skin treatment may be set to 20% higher level.

For another example, when the information on a skin color is input as the biometric information, the patient information providing unit 110 may provide the information on the skin color to the treatment information generating unit 120 which is to be describe below, but separately, the patient information providing unit 110 may generate the first skin information on a skin type, to which a skin type of the patient corresponds, among skin types classified according to Fitzpatrick skin type by using the information on the skin color.

When the skin type classified according to Fitzpatrick skin type is close to a dark skin color, the laser is absorbed well, so that the laser radiation intensity may be set to be low, and when the skin type classified according to Fitzpatrick skin type is close to a light skin tone, the laser is not absorbed well, so that the laser radiation intensity may be set to be high.

Further, the patient information providing unit 110 serves to generate and provide the second skin information through an external separate device.

The second skin information is skin thickness information measured through at least one device of an ultrasonic photographing device, a Computed Tomography (CT) device, and a Magnetic Resonance Imaging (MRI) device, and the patient information providing unit 110 serves to provide information input through the devices in a wired/wireless method as the second skin information.

In addition, the patient information providing unit 110 serves to generate and provide the third skin information through an external separate device.

The third skin information is skin lesion information for each part obtained through the skin analysis through ultraviolet light or polarized light radiation, and the patient information providing unit 110 serve to provide information input through the devices in a wired/wireless method as the third skin information.

Accordingly, the patient information providing unit 110 may be formed of a biometric information providing unit (not illustrated) which receives and provides the biometric information of the patient, and a processed information providing unit (not illustrated) which generates and provides the first skin information analyzed based on the biometric information, the second skin information obtained by photographing the skin of the patient, and the third skin information obtained through the light radiation to the skin of the patient.

The patient information providing unit 110 provides the input biometric information, first skin information, second skin information, and third skin information to the treatment information generating unit 120.

The treatment information generating unit 120 receives the biometric information, the first skin information, the second skin information, and the third skin information from the patient information providing unit 110, and recognizes skin thickness, skin color, and skin lesion from the information and generates treatment information for treating the patient.

The treatment information is generated for each patient, lesion, and part, and is provided to the practitioner on the screen through the display unit 190 which is to be described below, and is provided as at least one information of hand piece information, cartridge information, pin type information, treatment intensity information, delay time information, cooling condition information, spot size information, frequency information, and period information which are determined to be most suitable for the selected patient, selected lesion, and selected part and is provided in a modifiable state.

When the treatment information for each patient, lesion, and part is generated based on the information received from the patient information providing unit 110, the treatment information according to the part is automatically calculated and provided as a default setting value on the screen of the display unit 190 viewed by the practitioner, but the treatment information may be changed or corrected according to experience and skill level of the practitioner, so that the treatment information is provided in the modifiable state.

The treatment information generating unit 120 displays the generated default setting value, and when a separate correction command is not input from the practitioner, the treatment information generating unit 120 drives a treatment device A connected with the treatment information providing device 100 with the corresponding default setting value, and when a correction command for the default setting value is input from the practitioner, the treatment information generating unit 120 drives the treatment device A connected with the treatment information providing device 100 according to the changed setting value according to the corresponding correction command.

In the meantime, when the treatment progresses according to the default setting value generated in the treatment information generating unit 120 or the treatment progresses after the default setting value generated in the treatment information generating unit 120 is partially corrected according to the preference of the practitioner, the progress information input unit 130 serves to receive progress information about the completed treatment.

The progress information is the concept including primary progress information based on satisfaction input from the practitioner or the patient after the treatment is completed, and secondary progress information based on a change in the skin condition input from the practitioner or the patient before an additional treatment after a predetermined time.

That is, when the treatment is completed, the practitioner may allow the progress information on the treatment performed by the practitioner to be input through the progress information input unit 130 as the primary progress information, and the progress information may be used when the practitioner treats another patient or treats the same patient at the next time.

Further, the patient may also allow the progress information on the satisfaction of the received treatment to be input through the progress information input unit 130 as the primary progress information, and may input treatment satisfaction including an evaluation on the practitioner through the primary progress information.

In the meantime, changes in the skin condition may be more markedly changed, not immediately after the treatment is completed, but immediately before the subsequent treatment is performed. Accordingly, the practitioner may allow the progress information on the previously performed treatment is performed to be input immediately before the subsequent treatment through the progress information input unit 130 as the secondary progress information, and the practitioner may use or refer to the secondary progress information for this treatment, which is the next round of the treatment.

Also, the patient may allow the change in the skin condition according to the received treatment to be input through the progress information input unit 130 as the secondary progress information, and the practitioner may use or refer to the secondary progress information for this treatment, which is the next round of treatment.

In the meantime, the more important fact than the fact the practitioner uses the progress information at the time of the next round of treatment is that the progress information may be input for each patient, lesion, and part, and the treatment information providing device 100 may change the default setting value and reflect the changed default setting value to the time of the next round of treatment through the input progress information.

For example, the treatment information was generated to radiate a laser with power of 1.5 J to the area around the patient's right cheekbone and the treatment was performed according to the treatment information, and when the fact that the pain of the patient to the corresponding part is severe is input as the progress information, the treatment information may be updated or corrected so that a laser with power of less than 1.5 J is radiated to the corresponding part.

For another example, when it is determined that the level of spot removal in the area around the left cheekbone is lower than the area around the patient's right cheekbone, the treatment information generating unit 120 may have a new default setting value while the setting values for removing spots around the left cheekbone are increased compared to the existing treatment information.

As described above, the treatment setting values for the objective treatment may be provided as the default through the information of the patient, and the default setting values may be changed and provided again according to the satisfaction of the practitioner and the patient or the progress of the skin condition.

The internal access processing unit 140 is provided for the purpose of processing log-in of the practitioner accessing the treatment information providing device 100. The internal access processing unit 140 may be provided to enable log-in processing in a general ID/password method.

In addition, the internal access processing unit 140 may be provided to enable log-in processing by using the biometric information of the patient only when the patient is together in order to prevent unauthorized modification of the treatment information by the practitioner.

For example, the internal access processing unit 140 may be provided with an iris recognition module and the like to enable log-in processing to allow read of the treatment information of the practitioner through iris recognition of the patient.

The remote access processing unit 160 may be provided for the purpose of processing log-in through an external terminal when the patient attempts to log-in from the patient terminal 200. The remote access processing unit 160 may also process log-in by using the biometric information, such as fingerprint and iris, of the patient.

The storage unit 170 may serve to store programs for driving the entire treatment information providing device 100 for providing treatment information, store the patient information, the treatment information, the progress information, and the like, or store the progressed patient information, treatment information, and progress information, and the like. The storage unit 170 may serve to store programs for generally driving the treatment information providing device 100 for providing treatment information, store the patient information, the treatment information, the progress information, and the like, or process and store the patient information, the treatment information, the progress information, and the like.

As a matter of course, the storage unit 170 may also temporarily store the information and then process the information so that the information is separately stored in the external server 300.

The communication unit 180 may be provided for the purpose of allowing the primary progress information and at least one secondary progress information stored in the storage unit 170 or the server 200 to be shared with an external treatment information providing device (not illustrated) used by another practitioner.

Through this, the practitioners are capable of sharing the progress and the like when the treatment is performed according to the treatment method based on the treatment information, so that the information may be used as reference when other practitioners perform the treatment.

The display unit 190 serves to display the input patient information, treatment information, progress information, and the like on the screen and provide the practitioner or the patient with the information. The display unit 190 may be provided with a touch screen-type display, and in this case, the display unit 190 may serve to directly receive basic information, such as height and weight, as the patient information of the user, and may be operated as an input interface for the purpose of modifying or changing, by the practitioner, the treatment information that is the setting value set as the default.

The control unit 150 serves to control the general configurations of the treatment information providing device 100 for the series of operations for providing the treatment information, and serves to transceive information with the patient terminal 200 through the external server 300 and allow the treatment device A to be driven according to the treatment information.

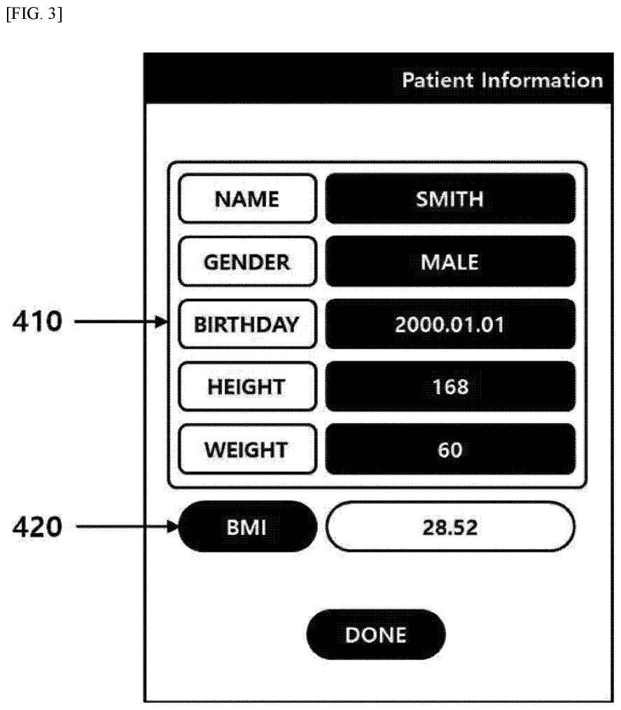

FIG. 3 is a diagram illustrating a screen in which patient information is provided according to the exemplary embodiment of the present invention.

As described above, the patient information may be displayed on the screen through the display unit 190, and may be input through a separate external input interface or a touch screen function of the display unit 190 itself.

Herein, in case of the separate external input interface, the display unit 190 may be provided with an interface for a direct input through a keyboard, a mouse, and the like, and may be provided with a data communication interface through a connection with an automatic weight/height measuring device.

When the basic biometric information 410 is input as the patient information as described above, the treatment information providing device 100 may measure a BMI 420 by using the biometric information 410, and calculate a default setting value for the skin treatment as first skin information suitable for the patient through the measured BMI 420.

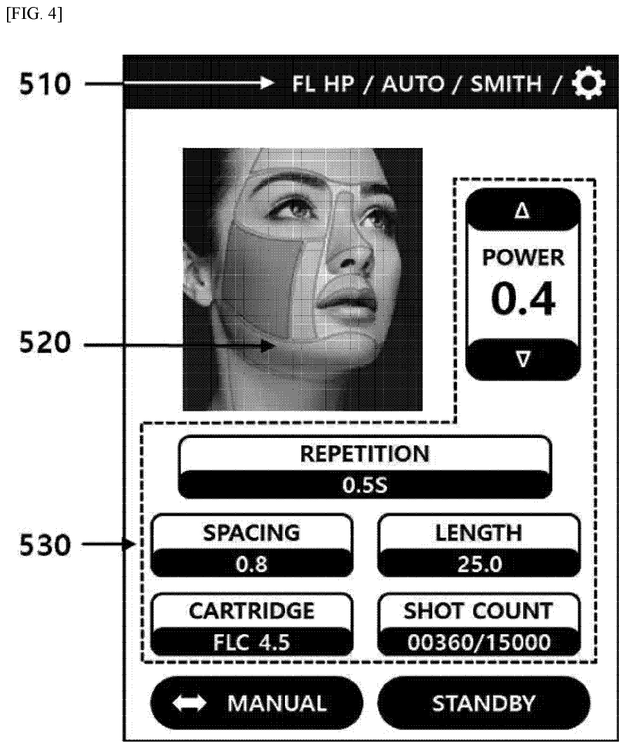

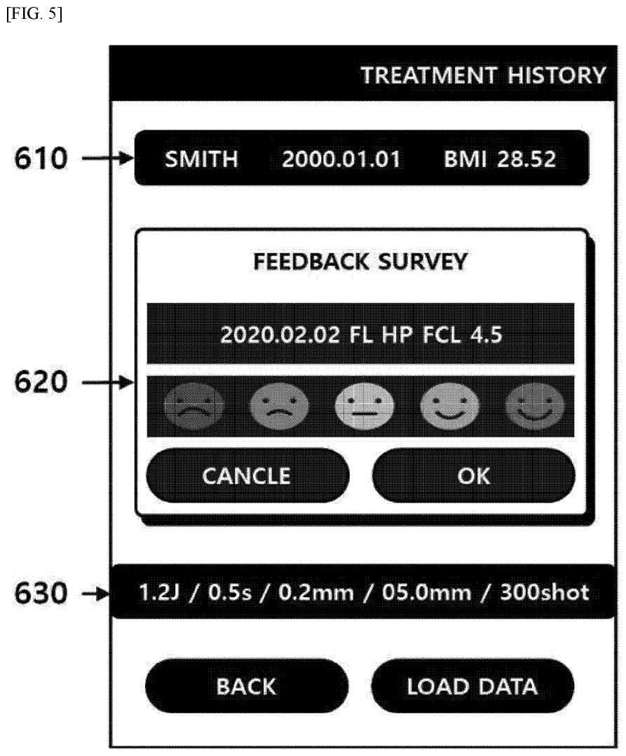

FIG. 4 is a diagram illustrating a screen in which treatment information is provided according to the exemplary embodiment of the present invention.

As described above, the treatment information may be displayed on the screen through the display unit 190, and may be corrected by the practitioner through the touch screen function of the display unit 190 itself.

The basic information of the patient and the basic information for the treatment among the treatment information may be provided to the topmost upper end of the screen in the form of a status bar 510. That is, according to the illustrated example, it can be seen that the present treatment information is the treatment information for treating a person named “SMITH” through an AUTO mode by using a handpiece called FL HP.

Further, the screen for selecting a treatment part of the patient among the treatment information may be provided in the form of a part selectable window 520 at the left center portion. That is, according to the illustrated example, it can be seen that the treatment information for treatment on the area around the cheekbone is provided.

Of course, these are only illustrative items for convenience of the description, and design changes may be made for how to divide the face part and what items are displayed when the face part is selected unlike the illustration.

Further, the screen for displaying and modifying the treatment setting values and correcting the treatment setting values for treatment on the treatment part of the patient among the treatment information may be provided in the state of a display bar 530 including multiple factor-based setting values from the right center portion to the lower end. That is, according to the illustrated example, it can be seen that the 25 mm ultrasonic waves having 0.8 mm spacing are radiated while repeating every 0.5 S with power of 0.4 J, and the ultrasonic waves are radiated by using a cartridge of FLC 4.5, and 360 shots of the 15,000 shots are currently radiated.

As described above, the factor-based setting values may be provided to the practitioner as the default based on the patient information, and the practitioner may correct the default setting values according to his/her skill level and experience and perform the treatment.

As described above, it is possible to promote the objectivity of the treatment by providing the automated setting value based on the patient information and reflect the expertise of the practitioner by enabling the modification of the automated setting value.

FIG. 5 is a diagram illustrating a screen in which progress information is input according to the exemplary embodiment of the present invention.

As described above, the progress information may be displayed on the screen through the display unit 190, and may be input through a separate external input interface or input through the touch screen function of the display unit 190 itself. Further, when the progress information is input by the patient, the progress information may be input through the application program executed in the patient terminal 200 possessed by the patient.

As described above, the progress information may be divided into the primary progress information input immediately after the treatment, and the secondary progress information input immediately before the next round of the treatment. In the illustrated example, the case in which the primary progress information is input is assumed for convenience of the description.

First, basic information of the patient for inputting the primary progress information may be displayed in an upper bar 610. Further, information on a setting value used in the already-completed treatment may be displayed in a lower bar 630.

The practitioner and the patient may input satisfaction of the treatment as the primary progress information through the middle window 620 without misunderstanding with other treatments by referring to the information of the upper bar 610 and the information of the lower bar 630.

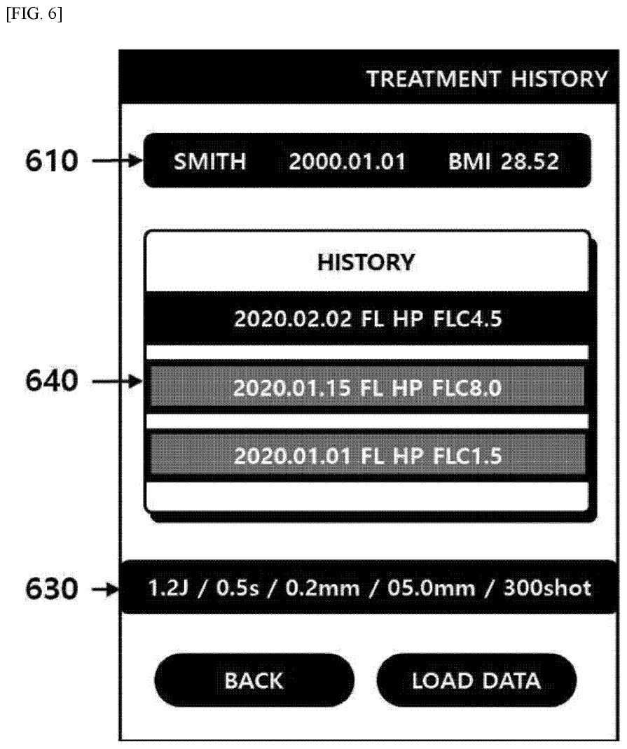

FIG. 6 is a diagram illustrating a screen in which a treatment history is recorded according to the exemplary embodiment of the present invention.

A history for the treatment may also be displayed on the screen through the display unit 190, and may be displayed on the screen of the patient terminal 200 through the application program executed in the patient terminal 200 possessed by the patient.

The histories for the treatment that has been performed progressed so far are displayed on the middle window 640 of the screen by date. When a specific history is selected among the treatment histories, the basic information of the patient and the information on the setting values used in the corresponding treatment may be displayed in the upper bar 610 and the lower bar 630 as described with reference to FIG. 5.

By providing the treatment history, the practitioner may reflect the treatment history to a next treatment through reading of the past record and enable a successful treatment, and the patient may receive objective information and increase reliability for the treatment.

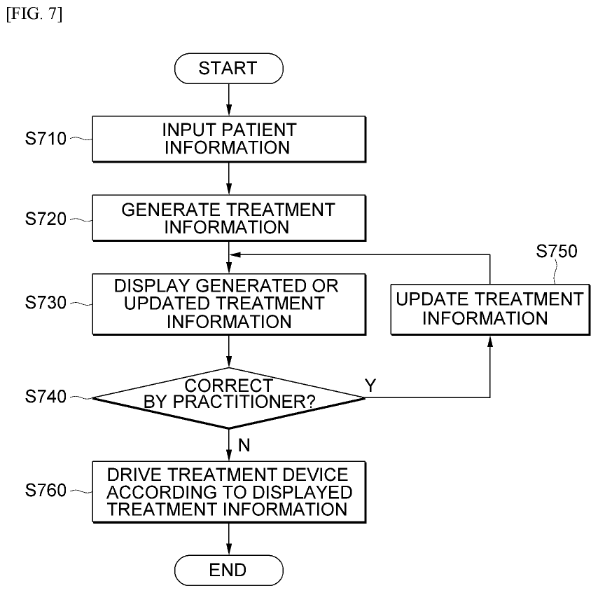

FIG. 7 is a diagram illustrating a method of providing treatment information for a skin care treatment according to an exemplary embodiment of the present invention.

The method of providing treatment information according to the present exemplary embodiment generates treatment information based on input patient information and provides a practitioner with the generated treatment information, and provides the treatment information in the state where the treatment information is modifiable according to a skill level or expertise of the practitioner, in order to provide a skin care treatment in a more objective state by preventing the treatment being performed under different conditions for each practitioner or according to a condition of the practitioner.

To this end, when patient information is input (S710), the treatment information providing device 100 generates treatment information based on the input patient information (S720), and displays the generated treatment information on a screen (S730).

After the treatment information is displayed on the screen (S730), when there is no other modification by a practitioner (N in S740), the treatment information providing device 100 makes a treatment device be driven according to the displayed treatment information (S760).

However, after the treatment information is displayed on the screen (S730), when the modification of the treatment information of the practitioner is input (Y in S740), the treatment information providing device 100 updates the displayed treatment information (S750) and displays the updated treatment information on the screen (S730).

After the updated treatment information is displayed on the screen (S730), when there is no additional modification by the practitioner (N in S740), the treatment information providing device 100 makes the treatment device be driven according to the displayed treatment information (S760).

Therefore, it is possible to provide the skin care treatment in a more objective state by preventing the treatment from being performed under different conditions for each practitioner or according to a condition of the practitioner, and it is further possible to perform a safe and effective treatment by preventing misuse of medical technology due to the misjudgment of the practitioner.

1. A device for providing treatment information, the device comprising:
a patient information providing unit configured to provide patient information;
a treatment information generating unit configured to generate treatment information based on the patient information;
a progress information input unit configured to receive progress information for the treatment when the treatment is completed based on the treatment information; and
a control unit configured to update the treatment information based on the progress information.

2. The device of claim 1, wherein the patient information providing unit includes:
a biometric information providing unit configured to receive and provide biometric information of a patient as the patient information; and
a processed information providing unit configured to generate and provide first skin information that is analyzed based on the biometric information, second skin information obtained by photographing a skin of the patient, and third skin information obtained by radiating light to the skin of the patient as the patient information.

3. The device of claim 2, wherein the first skin information is at least one of skin thickness information classified according to a Body Mass Index (BMI) in the biometric information and skin color information classified according to Fitzpatrick skin type in the biometric information,
the second skin information is skin thickness information measured through at least one device among an ultrasonic photographing device, a Computed Tomography (CT) device, and a Magnetic Resonance Imaging (MRI) device, and
the third skin information is skin lesion information for each part obtained through a skin analysis through ultraviolet light or polarized light radiation.

4. The device of claim 3, wherein the treatment information generating unit generates the treatment information for treating the patient before a skin treatment based on the skin thickness information, the skin color information, and the skin lesion information, and
the treatment information is generated for each patient, lesion, and part, and is provided to a practitioner, and is at least one of handpiece information, cartridge information, pin type information, treatment intensity information, delay time information, cooling condition information, spot size information, frequency information, and period information which are determined to be most suitable for the selected patient, selected lesion, and selected part and is provided in a modifiable state.

5. The device of claim 4, further comprising:
an internal access processing unit configured to process log-in of the practitioner; and
a remote access processing unit configured to process log-in using the biometric information of the patient through an external terminal.

6. The device of claim 1, wherein the progress information includes primary progress information based on satisfaction input from the practitioner or a patient after the treatment is completed, and secondary progress information based on a skin state change input from the practitioner or the patient before an additional treatment after a predetermined time elapses.

7. The device of claim 6, wherein the treatment information is generated for each patient, lesion, and part and is provided to a practitioner, and is at least one of handpiece information, cartridge information, pin type information, treatment intensity information, delay time information, cooling condition information, spot size information, frequency information, and period information which are determined to be most suitable for the selected patient, selected lesion, and selected part and is provided in a modifiable state,
the primary progress information and the secondary progress information are input for each patient, lesion, and part, and
the control unit updates the treatment information for each patient, lesion, and part based on the input primary progress information and the input secondary progress information and provides the updated information in a modifiable state for the additional treatment.

8. The device of claim 6, further comprising:
a storage unit configured to store the primary progress information and at least one secondary progress information continuously generated when the additional treatment is repeated; and
a communication unit configured to allow the primary progress information and at least one secondary progress information stored in the storage unit to be shared with an external treatment information providing device used by another practitioner.

9. A system for providing treatment information, the system comprising:
at least one treatment information providing device configured to generate treatment information based on patient information, receive progress information for the treatment, update the treatment information, and provide the updated treatment information;
a server configured to store and update the patient information, the treatment information, and the progress information received from at least one treatment information providing device; and
a patient terminal provided to generate the progress information by accessing the server by using biometric information in the patient information.

10. A method of providing treatment information, the method comprising:
receiving patient information;
generating treatment information based on the patient information;
receiving progress information for the treatment when the treatment is completed based on the treatment information; and
updating the treatment information based on the progress information and providing the updated treatment information.
